(19) United States
(12) Patent Application Publication
(10) Pub. No.: US 2005/0103132 A1
(43) Pub. Date: May 19, 2005

(54) Method for carrying out thickness and hardness measurements on test bodies and carousel and wiper unit therefor

(76) Inventors: Martin Bracher, Alchenstorf (CH); Hans-Peter Studer, Muhledorf (CH)

Correspondence Address: SHAW PITTMAN;IP GROUP, 1650 TYSONS BOULEVARD, SUITE 1300, MCLEAN, VA, 22102, US

(21) Appl. No.: 10/980,244

(22) Filed: Nov. 4, 2004

(30) Foreign Application Priority Data
Nov. 14, 2003 (CH) 01958/03

(51) Int. Cl. G01N 33/15; G01B 11/00; G01B 11/06

(52) U.S. Cl. 73/866; 356/630; 356/614

(57) ABSTRACT

The body mass of the test body is optically determined in the holding station (1). Oblong test bodies are placed in grooves (13) and cylindrical test bodies in V-shaped slots (14). It is therefore possible to prepare optimally for the optical measurement of the thickness by knowing the geometry of the test body, and for the hardness measurement by presetting the jaws (62, 63) so that the individual steps can be carried out more rapidly in succession. A larger number of test bodies can therefore be tested in a shorter time.

The invention relates to a method for carrying out measurements on test bodies, in particular to a method for measuring the thickness of tablets and to a carousel for a device for carrying out the thickness measuring method. The invention furthermore relates to a device for measuring the hardness of tablets and to a wiper unit for a device for carrying out the hardness measuring method.

In the prior art for testing tablets, it is necessary to carry out various working steps with a view to measurements of body properties of such test bodies. These include, in particular, thickness measurement, weight measurement and hardness measurement.

Thickness measurement is a difficult technical field, since there are a multiplicity of different tablet shapes which are sometimes provided with a separating gap or a marker. Tablet thickness testers with a manual plunger are therefore used, in which case the user himself or herself has to find the appropriate thickness measuring point on the sample. It is also necessary to test gelatinous tablets, for which these contact measuring methods are so far unsuccessful or give unreliable results.

From the testing of tablets in other technical fields, such as the nuclear field, EP 0 528 197 for example discloses an optical recording system in which the tablets, which are in the form of small cylinders, are moved past a slot while they rotate, the shadowing and scattering properties being closed onto the outline of the tablets. Owing to the different material composition of tablets to be orally consumed, such a device cannot be used for testing these.

U.S. Pat. No. 4,884,463 describes a carousel with various workstations, in particular a weighing station, a thickness measuring station and a hardness measuring station. The carousel is subdivided by individual radially arranged recesses, for example five recesses for the three measuring stations as well as a delivery station and a discharging station. The recesses are bounded towards the middle of the carousel by an abutment wall aligned along the circle, a wall leading to the opening of the recess is essentially aligned radially and the opposite wall is a concave wall comprising two straight segments, which form a laterally aligned V-shape. The three measuring stations are arranged in conjunction with the carousel in such a way that they give rise to mechanical constraints, which make quantitative or qualitative improvement of the measurement difficult. The device is a revolver device in which all the functions are arranged at a position around the rotation axis.

EP 0 983 495 describes another method for carrying out a hardness test on a test body, in which the position of the test body is established in an optical, electronic or acoustic way by determining the preferential axis. The instrument which carries out the hardness test is subsequently rotated so that the pressure piston acts on the test body in the said preferential direction. Adjustment for only one measuring station is disadvantageous owing to the slowness of the measuring method.

On the basis of this prior art, it is an object of the invention to improve a method and a device of the type mentioned in the introduction, so that the relevant testing can be carried out more reliably and more rapidly, with the user who is preparing for the test being optimally assisted in this.

For a thickness measuring method, this object is achieved by the features of claim 1. A carousel of a device according to the invention is characterized by the features of claim 5.

By knowing the geometry of the test body, the optical measurement of the thickness and the hardness measurement can be prepared for optimally by presetting the jaws. At the same time, the test body can be disposed of more rapidly after the hardness measurement.

Since the body mass of the test body is determined optically in the holding station, the thickness or hardness measurement can be prepared for optimally so that the individual steps can be carried out more rapidly in succession. A larger number of test bodies can therefore be tested in a shorter time.

Further advantageous exemplary embodiments are characterized in the dependent claims.

The invention will now be described in more detail by way of example with reference to the drawings, in which:

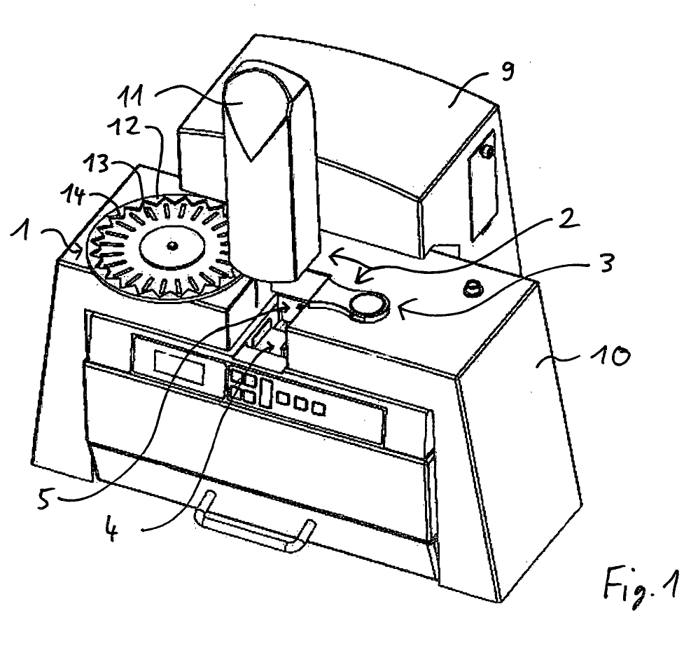

FIG. 1 shows a perspective overall view of a device for testing test bodies, in particular tablets or pills, with five stations,

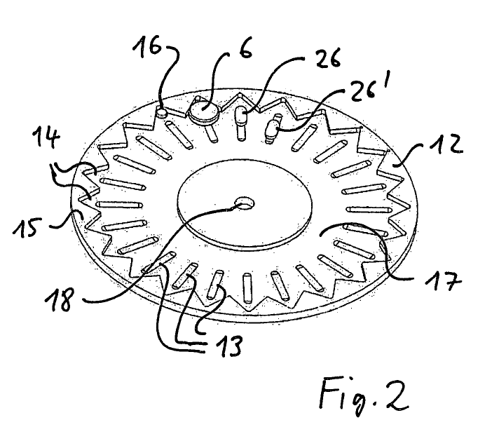

FIG. 2 shows a perspective view of a carousel for holding the test bodies at a loading station,

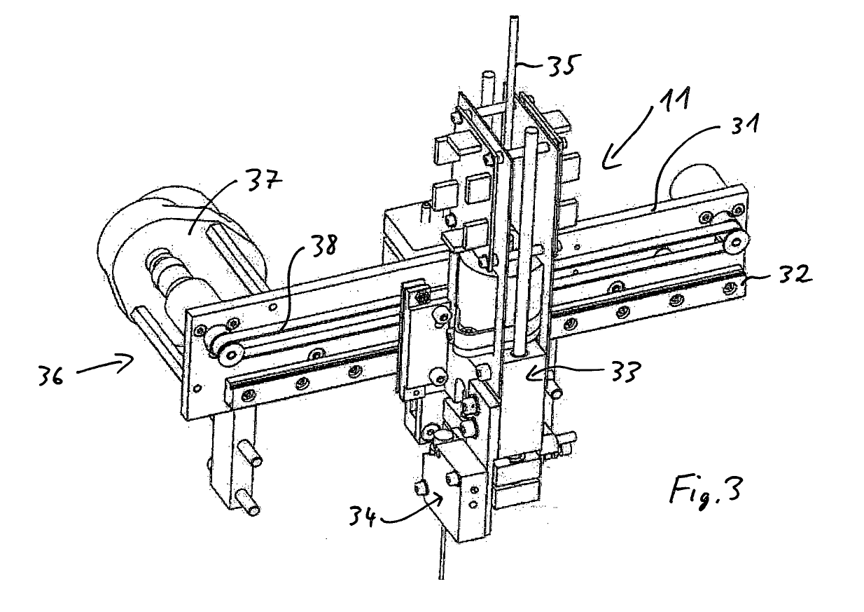

FIG. 3 shows a perspective view of a combined displacing and measuring unit for moving and measuring the test bodies between the individual stations, or at a thickness measuring station, and

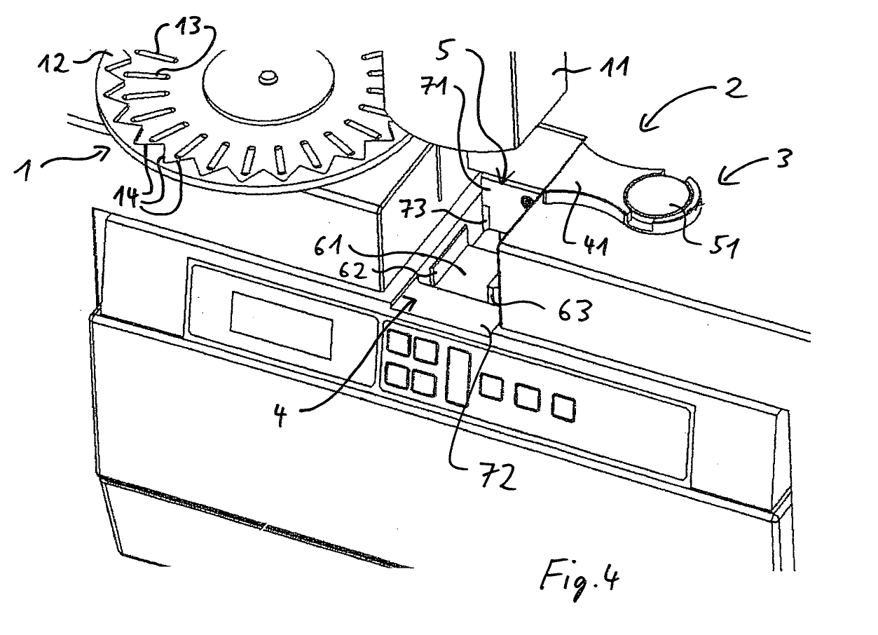

FIG. 4 shows an enlarged partial perspective view of the device according to FIG. 1 for testing test bodies, in particular tablets or pills, with five stations.

FIG. 1 shows a perspective overall view of a device 10 for testing test bodies, in particular tablets or pills, with five stations. These are the loading station 1, the thickness measuring station 2 which comprises a mobile arm 11, the weighing station 3, the hardness measuring station 4 and the discharging station 5. The connecting element of the stations is the mobile arm 11.

The loading station 1 has a carousel 12 with radial grooves 13 and V-shaped holding locations 14 in extension of the said grooves 13. The configuration and mode of action of the carousel 2 are defined more accurately in FIG. 2 and in conjunction with the description of this figure.

The mobile arm 11 is fastened in a mobile fashion to the frame body 9 of the device 10. The configuration and mode of action of the mobile arm 11 are defined more accurately in FIG. 3 and in conjunction with the description of this figure.

FIG. 2 shows a perspective view of a carousel for holding the test bodies 6, 16 at a loading station 1. Two different sizes of test body 6 and 16 are represented which, for example, are cylindrical tablets with two different diameters in this case. The carousel 12 is round and has a depressed loading surface 17. Radial elongate indentations 13, in which elongate test bodies 26 can be placed, are preferably provided on the surface 17. These may, for example, be an administrable form of medications, which are referred to as capsules or oblongs. The capsules are usually an axisymmetric cylindrical body with hemispherical ends. Owing to the grooved shape of the indentations 13, such capsules can easily be placed always at the same radial position on the carousel 12. The exact position can be established by the measuring procedure described below, and it is therefore possible to discriminate between position displacements, for example as represented with the oblongs 26 and 26′ in FIG. 2. The grooves 13 may also be wider in the central part in order to form a stable, deeper bearing point.

The cylindrical test bodies 6 or 16 can be placed in one of the holding locations 14, of which there are twenty-four in this case. This can be done by simple placement on the flat surface 17 and subsequent displacement against the V-shaped holding locations 14. This makes it possible to arrange different sizes of test bodies 6, 16 centred on the carousel, in so far as the median plane of the test bodies 6, 16 coincides with the axis which passes through the groove middle of the grooves 13 and the apex of the V-shaped holding locations. The test bodies 6, 16 remain at the placement position since the elevated edge 15 prevents the test bodies 6, 16 from falling out.

The carousel 12 has a central hole 18, so that it can be fitted onto a driven shaft. This makes it possible to load the carousel 12 in advance and subsequently fit it onto the drive. The number of recesses 14/grooves 13 may also be selected differently, the V-shaped holding location may also be U-shaped, and an aligned arrangement of a large number of test bodies 6, 16 per batch is possible, which may even simultaneously involve a wide variety of test bodies 6, 16, 26 or the like. Generally, however, a batch will be equipped with respectively identical test bodies.

FIG. 3 shows a perspective view of the combined displacing and measuring unit, referred to as a mobile arm 11 in FIG. 1, for moving and measuring the test bodies between the individual stations or at a thickness measuring station. The base plate 31 is integrated and fastened in the frame body 9. Guide elements, such as a guide rail 32 in this case, are provided on this base plate 31. The displacing unit 33 and the measuring unit 34 are guided on the base plate 31 and, in particular, via the guide rail 32.

The displacing unit 33 is a conventional suction instrument in this case, with a downwardly directed soft opening which can be lowered along the movement direction, defined for example by the shaft 35, in the direction of a test body 6, 16 in order to pick it up by generating a reduced pressure in the region of the opening.

The measuring unit 34 is arranged separately from this displacing unit 33, that is to say the measuring unit 34 does not move with the displacing unit 33 in the direction of the axis defined by the shaft 36. The measuring unit 34 may, however, also move with the displacing unit 33 in this direction, in which case the measurements to be taken by the measuring unit are then preferably carried out at the height position where the mobile arm is located with the greatest height difference from the carousel 12 and other elements.

A displacing instrument 36 which essentially operates horizontally is provided on the base plate, with a drive 37 driving a pulley belt 38 which is tensioned horizontally between the two end regions of the plate 31. A toothed chain may also be used, Alternatively, a stepper motor could also be integrated in the mobile arm 11. What is essential is that the mobile arm 11 can be moved essentially horizontally with the displacing unit 33 (via the suction head for test bodies) and the measuring unit 34 (in this case a laser sensor). This means that the two units 33 and 34 are arranged over the region of the carousel 12 represented on the right in FIG. 1 and can travel over it in the radial direction, and that the two units 33, 34 can furthermore travel over the measuring stations 4 and 5 placed below this position to the bearing surface of the measuring station 2 and the weighing surface of the station 3, so that the test bodies 6, 16, 26 held by the suction head can be moved between all the stations 1, 2, 3, 4, 5. Devices which involve one of the different contactless measuring methods, such as ultrasound or laser reflection sensors, may be used for the measuring unit.

The measuring unit 34 is, in particular, a laser sensor (in this case the laser distance sensor OADM 12I6430/S35A from Baumer Electric) which, when used over the loading station 1, emits a laser beam essentially at right angles to the surface 17 of the carousel 12, which is reflected by the corresponding surfaces (surface 17; test body 6, edge surface 15). By recording the back-reflected and back-scattered signal, it is possible to determine the distance of the reflection point. The mobile arm 11 is moved along over the tablet, that is to say in the direction from the apex of the V-shaped holding location 14 flush with the mid-plane of the groove 13, in other words along the radius. The respective mid-profile is scanned by virtue of the centring of the test body 6, 16 (at 14) or the ovaloid or oblong tablet (at 13). The diameter or length of the tablet can be determined directly in this way. This knowledge can also be used in the hardness measuring station 4. Any transverse furrows or impressions can be automatically detected by this method. This is advantageous because the suction head 33 can thus be placed next to such features, on a flat or only slightly rounded section of a tablet. This prevents the suction from being broken and/or the tablet from entering an incorrect position by the tablet held by suction slipping over such a furrow or impression.

If only one direction (along the radius) is being scanned, then it is necessary to ensure that any furrows transverse or at least inclined to the scanning direction are covered. This requirement is obviated by two-dimensional scanning. This may also be achieved by moving the laser sensor 34 to a particular place at a defined distance from the middle of the carousel 12 before the final rotation of the carousel into a new position, so that the test body 6, 16, 26 is transversely scanned by the laser sensor when the carousel 12 subsequently rotates.

The use of a light sensor in general, and of a laser sensor in particular, also allows straightforward encoding of the carousel 12 by either a particular pattern of holes or a particular reflectivity in different regions etc. By scanning a position for a test body 6 in the carousel 12, the light sensor can also establish whether or not there is actually a test body 6, 16, 26 in a given holding location 14, or whether the recording step can be skipped for this position if the groove 13 is indeed empty. With oblong test bodies, it is essential to place them centrally in the groove 13; the actual radial position in the groove 13, however, is unimportant since the light sensor will find a suitable recording position by sampling or scanning. By sampling the oblong test bodies 26 in the radial direction, it is already possible to establish their length on the test carousel 12, so that the middle of the test body 26 can be established directly by a calculation.

The suction head 33 touches the surface of a test body 6, 16, 26, while applying a reduced pressure continuously or only during the displacement to be carried out. The mobile arm 11 can then have its height adjusted and be moved in the direction along the rail 32. This moves the test body in the direction of the thickness measuring station 2 or the weighing station 3. The processing sequence of the stations 2, 3 is not essential, except when having to carry out a thickness measurement on special test bodies which do not have a surface, and which therefore tilt if they are not held laterally. In this case, a prop is used which is placed on the thickness measuring platform. This prop has a recess matched to the tablet, which prevents the tablet from tilting in the course of the thickness measurement.

In the exemplary embodiment represented here, it is assumed that the suction head first places the specimen 6, 16, 26 essentially centrally on the flat measuring platform 41 of the thickness measuring station 2. The light sensor is then moved over the entire test body 6, 16, so that it is possible to establish a reference value for the bottom in the region where the light is incident on the measuring platform, and the height of the highest point above the bottom of the measuring platform 41, which corresponds accurately to the maximum thickness of the specimen, by minimization of the distance. As an alternative, before placing the specimen or after lifting the specimen off the measuring platform 41 again, the sensor may be moved to the position where the highest point was measured, so as to determine the distance from the measuring platform 41 separately at this position.

At the same time, by sweeping the measuring window on the measuring platform 41 with a scattered-light measurement, the controller can also establish whether any detached tablet residues or other particles have contaminated the measuring platform 41 and falsified the measurement.

By applying suction to the test body again, it is subsequently lifted off the measuring platform 41 and moved to the weighing station 3, where it is placed on the weighing platform 51 which is connected to a balance arranged underneath.

When suction is applied for the last time to the test body 6 after the weighing process, it is placed on the hardness test platform 61 of the hardness measuring station 4. Here, the jaw 62 is moved towards the abutment 63 and compresses the test body 6 during the measuring step. The length measured beforehand on the carousel 12 is then advantageously transmitted to the hardness measuring station 4, so that the jaw 62 can already be moved close to the contact point before the test body 6 is deposited, in order for the hardness measurement to be carried out directly after bringing the jaws together through a small distance. This procedure is also possible when a second jaw is provided instead of an abutment 63.

There are tablet residues on the hardness testing platform 61 of the hardness measuring station 4 after the hardness measurement. These are wiped away by the wiper 71, for example into the opening 72, so that the hardness measuring station 4 is free for the next measurement.

1. Method for carrying out measurements on test bodies, the measurement belonging in particular to the group: thickness measuring methods, weighing methods and hardness measuring methods, which can be carried out in an appropriate measuring station (2, 3, 4), with a holding station (1) which has a carousel (12) that holds the specimens (6, 16) in a defined position, characterized in that a mobile light sensor (34) is provided, the light signal of which can be displaced across the test body (6, 16) at least in the radial direction relative to the carousel (12) in order to determine the presence and/or position and/or length and/or diameter, the carousel (12) having means (13, 14) for positioning the principal axis of the test body (6, 16) on a radial path, and in that a lifting means (34) is provided in a defined relation with the light sensor (34) and can be respectively used to take a test body (6, 16) off the carousel (12) in order to place it in one of the said measuring stations (2, 3, 4) in a defined way.

2. Method according to claim 1, characterized in that the thickness measuring station (2) has a measuring platform (41), and in that the said light sensor (34) determines the relative distance of the measuring platform (41) from the light sensor (34) and, by scanning the test body (6, 16) via the median line, the latter's minimum distance from the light sensor (34) so that the maximum thickness of the test body (6, 30 16) can then be determined by taking the difference between the two distances.

3. Method according to claim 1, characterized in that the weighing station (3) has a measuring platform (51) which is designed as a balance, and in that the weighing method step involves placing the test body (6, 16) on the measuring platform (51), recording the higher weight of the platform (51) and the test body (6, 16) due to the test body (6, 16) and removing the test body (16, 16) for delivery to another workstation (2, 4, 5).

4. Method for carrying out measurements on test bodies with at least one holding station (1) and a hardness measuring station (4) with the method steps according to claim 1.

5. Carousel (12) for a device for carrying out a thickness measurement according to claim 1, with holding locations (13, 14) arranged regularly near the circumference for the test bodies (6, 16), characterized in that each holding location (13, 14) has a radial indentation (13) for stably holding oblong test bodies and an abutment (14) centering on a radius line for cylindrical test bodies (6, 16).
